(19) United States
(12) Patent Application Publication
(10) Pub. No.: US 2012/0122896 A1
(43) Pub. Date: May 17, 2012

(54) 2,1,3-BENZOXADIAZOL DERIVATIVES FOR THE INHIBITION OF INFLUENZA A AND B VIRUS AND RESPIRATORY SYNCYTIAL VIRUS REPLICATION

(75) Inventors: Ulrich Kessler, Boppelsen (CH); Charlene Ranadheera, Winnipeg (CA)

(73) Assignee: PIKE PHARMA GMBH, Zurich (CH)

(21) Appl. No.: 13/318,763

(22) PCT Filed: May 7, 2010
(86) PCT No.: PCT/EP2010/056294

§ 371 Date: Feb. 2, 2012

(30) Foreign Application Priority Data
May 8, 2009 (EP) PCT/EP2009/055633

(51) Int. Cl. A61K 31/4245; A61K 31/52; C07D 473/38; A61P 31/14; C07D 271/12; A61K 31/426; A61P 31/16

(52) U.S. Cl. 514/263.2; 514/364; 548/131; 544/265

(57) ABSTRACT

A 2,1,3-benzoxadiazole compound as a medicament according to the invention is one of the following compounds: 4-[(4-methoxybenzyl)thio]-7-nitro-2,1,3-benzoxadiazole, 2-[(7-nitro-2,1,3-benzoxadiazol-4-yl)thio]ethyl 4-methoxybenzene-1-sulfonate, 4-[(4-methylphenyl)thio]-7-nitro-2,1,3-benzoxadiazole, 4-[(2,4-dichlorophenyl)thio]-7-nitro-2,1,3-benzoxadiazole, 2-[(7-nitro-2,1,3-benzoxadiazol-4-yl)thio]ethan-1-ol, 4-[(4-methylbenzyl)thio]-7-nitro-2,1,3-benzoxadiazole, 4-[(4-fluorophenyl)thio]-7-nitro-2,1,3-benzoxadiazole, 4-[(3-chlorophenyl)-thio]-7-nitro-2,1,3-benzoxadiazole, 2-[(7-nitro-2,1,3-benzoxadiazol-4-yl)thio]ethyl-4-methoxy-benzoate, 5-[4-(tert-butyl)-1,3-thiazol-2-yl]-2,1,3-benzoxadiazole, N-benzyl-4-nitro-2,1,3-benzoxadiazol-5-amine, 4-nitro-7-(phenylmethylsulfanyl)-2,1,3-benzoxadiazole, 4-nitro-7-(phenylmethylsulfonyl)-2,1,3-benzoxadiazole, 2-(hydroxymethyl)-5-[6-[(4-nitro-2,1,3-benzoxadiazol-7-yl)sulfanyl]purin-9-yl]oxolane-3,4-diole, or 2-[2-amino-6-[(4-nitro-2,1,3-benzoxadiazol-7-yl)sulfanyl]purin-9-yl]-5-(hydroxymethyl)oxolane-3,4-diol; or a physiologically tolerable salt, solvate, or physiologically functional derivative thereof. Said compounds are particularly advantageous for treating and/or preventing influenza type A and/or influenza type B infections in humans, mammals and/or birds, and for treating and/or preventing respiratory syncytial virus infections in humans, mammals and/or birds.

### FIELD OF THE INVENTION

The present invention relates to small molecules inhibiting the replication of influenza A and B virus and respiratory syncytial virus (RSV), and the use of such compounds for treating influenza A and B and RSV infections, in humans, mammals and birds.

### BACKGROUND OF THE INVENTION

Influenza viruses are negative-stranded RNA viruses that cause yearly epidemics as well as recurring pandemics, resulting in high numbers of human cases and severe economic burden. In addition to the well-known pandemic influenza A viruses (such as the 1918 “Spanish” flu or H₅N₁), both type A and B viruses contribute greatly to the annual recurring epidemics that cause the vast majority of human cases and medical cost. The WHO recommends an annual vaccination against circulating influenza A (FluA) and B (FluB) strains. However, current vaccines confer incomplete protection against epidemic influenza. To date, only the neuraminidase inhibitors oseltamivir (Tamiflu™) and zanamivir (Relenza™) are available as antiviral treatment against both virus types. However, there is a growing fear within the medical community about the rapidly growing emergence of influenza strains resistant to both drugs. The older adamantane drugs are not effective against FluB and the global spread of influenza viruses resistant to oseltamivir demonstrate the limitations of the neuraminidase inhibitors. A recent epidemiological survey in the U.S. found 98.5% of the H1N1 isolates tested resistant to oseltamivir.

Human respiratory syncytial virus (RSV) is a negative-sense, single-stranded RNA virus of the family Paramyxoviridae, and is the major cause for respiratory tract illnesses during infancy and childhood such as bronchiolitis and pneumonia. There is currently no vaccine available. Treatment is mainly limited to supportive care, including oxygen. Palivizumab (Synagis™) is used as a prophylactic drug in prevention of respiratory RSV infections for infants with a high risk of infection. Ribavirin has been used for treating RSV infections, but showed limited effectiveness.

Thus, new improved and alternative antiviral agents against both influenza A and B virus types and RSV are urgently needed.

### OBJECTS OF THE INVENTION

One object of the invention is to provide new, improved and/or alternative influenza and RSV antiviral compounds.

Another object of the invention is to obviate or mitigate disadvantages of influenza antiviral agents and RSV antiviral agents known from the state of the art.

These and other objects are achieved by a compound as a medicament, a compound for treating influenza type A and/or influenza type B and/or RSV infections in humans, mammals and/or birds, the use of a compound for the manufacture of a medicament for the treatment of influenza type A and/or influenza type B and/or RSV infections in humans, mammals and/or birds, and a pharmaceutical composition comprising such a compound, according to the independent claims. Advantageous embodiments are given in the dependent claims.

### SUMMARY OF THE INVENTION

The 2,1,3-benzoxadiazole compounds as a medicament according to the invention are: 4-[(4-methoxybenzyl)thio]-7-nitro-2,1,3-benzoxadiazole, 2-[(7-nitro-2,1,3-benzoxadiazol-4-yl)thio]ethyl 4-methoxybenzene-1-sulfonate, 4-[(4-methylphenyl)thio]-7-nitro-2,1,3-benzoxadiazole, 4-[(2,4-dichlorophenyl)thio]-7-nitro-2,1,3-benzoxadiazole, 2-[(7-nitro-2,1,3-benzoxadiazol-4-yl)thio]ethan-1-ol, 4-[(4-methylbenzyl)thio]-7-nitro-2,1,3-benzoxadiazole, 4-[(4-fluorophenyl)thio]-7-nitro-2,1,3-benzoxadiazole, 4-[(3-chlorophenyl)thio]-7-nitro-2,1,3-benzoxadiazole, 2-[(7-nitro-2,1,3-benzoxadiazol-4-yl)thio]ethyl-4-methoxybenzoate, 5-[4-(tert-butyl)-1,3-thiazol-2-yl]-2,1,3-benzoxadiazole, N-benzyl-4-nitro-2,1,3-benzoxadiazol-5-amine, 4-nitro-7-(phenylmethylsulfanyl)-2,1,3-benzoxadiazole, 4-nitro-7-(phenylmethylsulfonyl)-2,1,3-benzoxadiazole, 2-(hydroxymethyl)-5-[6-[(4-nitro-2,1,3-benzoxadiazol-7-yl)sulfanyl]purin-9-yl]oxolane-3,4-diole, and 2-[2-amino-6-[(4-nitro-2,1,3-benzoxadiazol-7-yl)sulfanyl]purin-9-yl]-5-(hydroxymethyl)oxolane-3,4-diol, as well as physiologically tolerable salts, solvates, or physiologically functional derivatives thereof.

The above defined compounds according to the invention are particular advantageous for treating and/or preventing influenza type A and/or influenza type B infections in humans, mammals and/or birds; as well as for treating and/or preventing respiratory syncytial virus (RSV) infections in humans, mammals and/or birds.

The compound according to the invention can be used for the manufacture of a medicament for the treatment and/or prevention of influenza type A and/or influenza type B infections in humans, mammals and/or birds, and/or for the treatment and/or prevention of respiratory syncytial virus infections in humans, mammals and/or birds.

A pharmaceutical composition according to the invention comprises a compound according to the invention. Advantageously such a composition comprises one or more excipients.

Surprisingly, it was found that compounds in accordance with the present invention are able to inhibit protein-protein interaction of the PA and PB1 subunits of the heterotrimeric viral RNA polymerase complex of both influenza virus types A and B, and thus are able to inhibit replication of influenza A and B virus. The viral polymerase subunit interaction domain turned out as an effective target for the new antiviral compounds, since correct assembly of the three viral polymerase subunits PB1, PB2 and PA is required for viral RNA synthesis and infectivity. Structural data for the entire trimeric complex is missing.

Based on the crystal structure of a truncated FluA PA in complex with the N-terminus of PB1 it was established that the crucial PA interaction domain of PB1 consists of a 3₁₀-helix formed by amino acids (amino acids 5-11). The domain is highly conserved and virus type specific among both, influenza A and B viruses.

An Enzyme-Linked ImmunoSorbent Assay (ELISA) based screening assay and other assays are used to prescreen compounds according to the invention that show antiviral activity against influenza A and B viruses. Since they are effective against both virus types, such compounds represent an attractive alternative to neuraminidase inhibitors. Therefore, the present invention represents a major step toward a sorely needed, near-universal medicament against influenza virus, and one which, due to its protein-protein interaction domain target, will likely be less susceptible to the emergence of drug-resistant strains for which influenza is well known.

Furthermore it was found that compounds according to the invention are also able to inhibit replication of respiratory syncytial virus (RSV).

Thus the compounds according to the invention can be used as a medicament, particularly as an influenza virus and/or RSV replication inhibitor and an influenza and/or RSV preventive/therapeutic agent, respectively.

The object, characteristics, and advantages of the present invention as well as the idea thereof will be apparent to those skilled in the art from the descriptions given herein. It is to be understood that the embodiments and specific examples of the invention described herein below are to be taken as preferred examples of the present invention. These descriptions are only for illustrative and explanatory purposes and are not intended to limit the invention to these embodiments or examples. Other aspects and features of the present invention will become apparent to those ordinarily skilled in the art upon review of the following description of specific embodiments of the invention in conjunction with the accompanying figures. It is further apparent to those skilled in the art that various changes and modifications may be made based on the descriptions given herein within the intent and scope of the present invention disclosed herein.

### DETAILED DESCRIPTION OF THE INVENTION

### Influenza Type A and B: Therapeutic Target

In the international patent application with the title “Influenza A and B virus replication-inhibiting peptides” No. PCT/EP2009/055632, filed on 8 May 2009, novel peptides containing for example amino acid sequences from both virus types A and B, are described. The content of said application is hereby included by reference in its entirety. Surprisingly, it was found that those novel peptides bind to PA subunits of both types of influenza A and B. Among said novel peptides, chimeric peptides, containing amino acid sequences from both virus types A and B, were identified which not only bind to both PA subunits, but also decrease the viral polymerase activity and the spread of virus in cell culture for both influenza A and B. In the following the findings concerning the binding of said novel peptides are described in order to further specify the inhibition target of the small molecules compounds according to the present invention.

It should be noted that all amino acids are preferably indicated by the IUPAC one letter code in the present application. Whenever three letter codes are used, they are also in accordance with IUPAC. The letter X is used to indicate a wildcard/variable or other amino acid at a certain position.

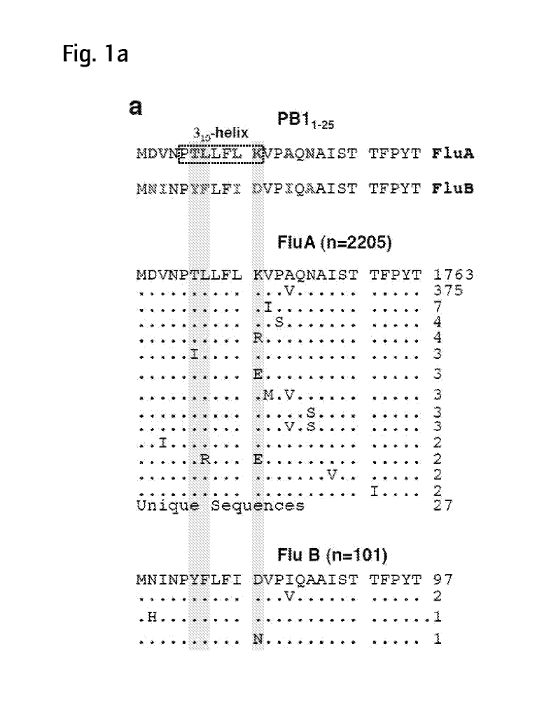

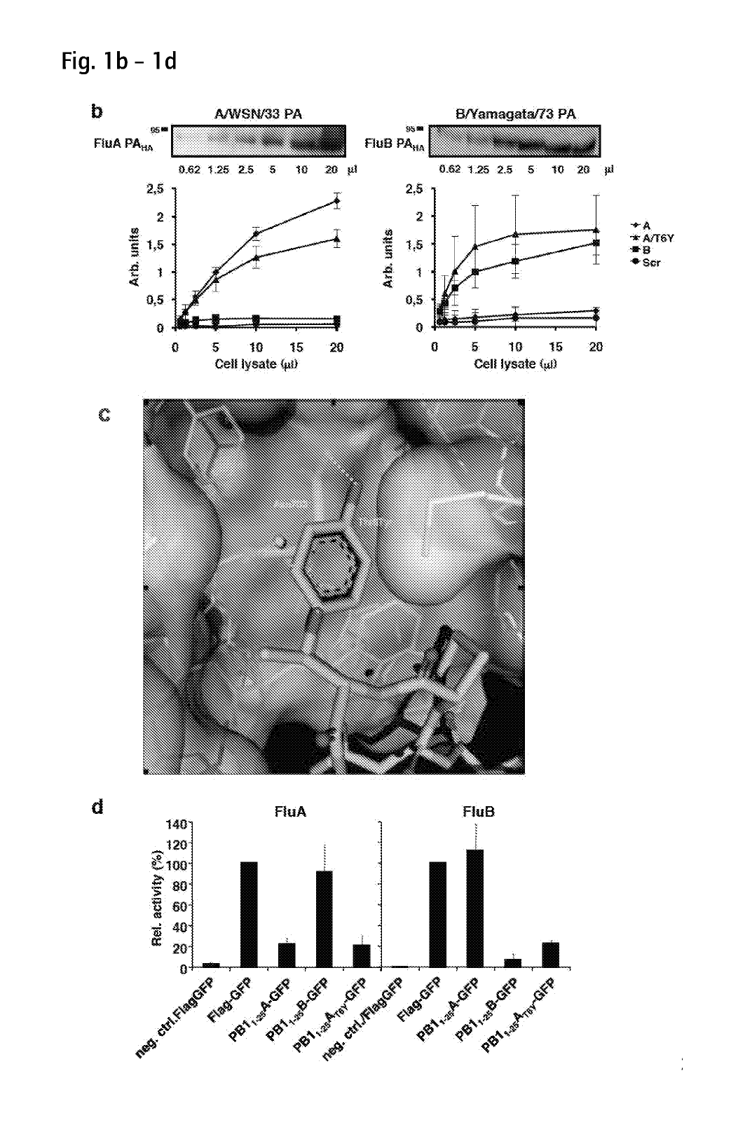

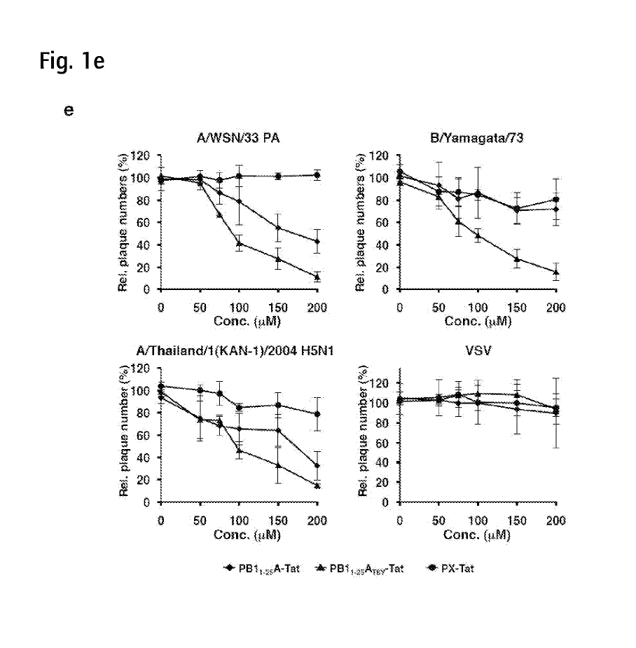

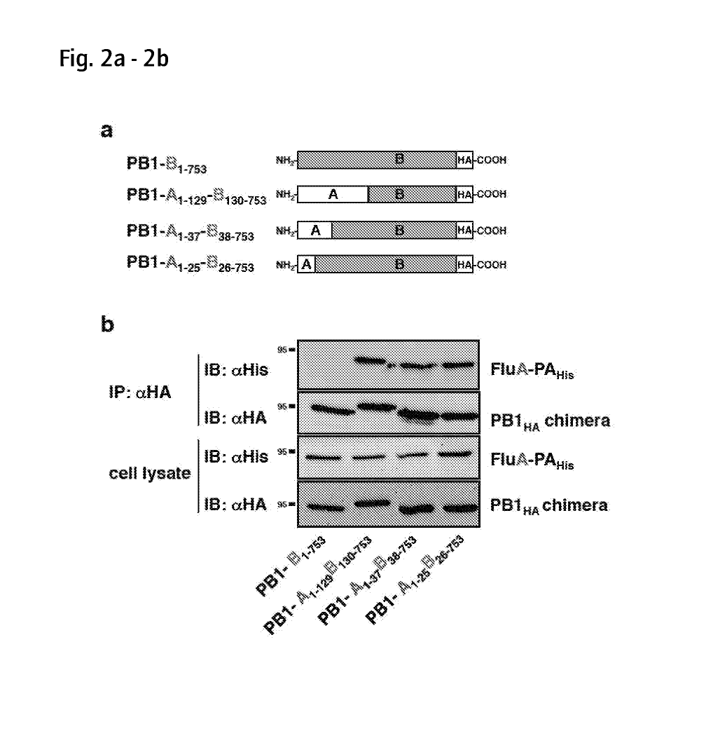

It has been found that the crucial PA interaction domain of PB1 consists of a 3₁₀-helix formed by amino acids X₅ to X₁₁. This domain is highly conserved and type-specific among both influenza A and B viruses (FIG. 1a). Additionally, FluB PB1 was able to bind to FluA PA when these 25 amino acids were exchanged with the FluA PB1 sequence (FIG. 2).

Table 1a shows the inhibitory concentrations of Flu/FluB-derived peptides determined by competitive ELISA. Competitor peptides (0.048 to 3000 nM) were mixed with cell extracts containing HA-tagged PA from either FluA or FluB. Table 1 lists 12 competitive peptides. The first peptide PB1₁₋₁₅A is the FluA wild type, the second row shows the FluB wild type. For the peptides of rows 3 to 8 letters indicate FluB specific amino acids. Rows 9 to 12 list further competitive peptides with amino acids at position 6 being neither FluA nor FluB specific. Standard deviation is indicated in parenthesis. Asterisks indicate highest concentrations of peptides used without reaching 50% inhibition. Further competitive peptides which are not listed in the table but have effectively reached 50% inhibition at low peptide concentrations are PB1₁₋₁₅A_{T6I}, PB1₁₋₁₅A_{T6L} and PB1₁₋₁₅A_{T6V}. Peptides with slightly lower inhibition activity are PB1₁₋₁₅A_{T6A} and PB1₁₋₁₅A_{T6M} which are also not shown in Table 1a.

TABLE 1a
inhibitory concentrations of FluA/FluB-derived
peptides determined by competitive ELISA

| | | IC₅₀ (nM) | |
|---|---|---|---|
| Competitive peptide | | PA (FluA) | PA (FluB) |
| PB1₁₋₁₅ A | MDVNPTLLFLKVPAQ | 43.32 (+/−5.31) | >3000* |
| PB1₁₋₁₅ B | . [symbol] .. [symbol] .. [symbol] ... [symbol] . | >3000* | 45.0 (+/−12.5) |
| PB1₁₋₁₅ A_{D2N, V3I, L14I} | [symbol] .......... [symbol] . | 6.69 (+/−1.73) | >3000* |
| PB1₁₋₁₅ A_{L10I, K11D} | ......... [symbol] .... | >3000* | >3000* |
| PB1₁₋₁₅ A_{D2N, V3I} | . [symbol] ............ | 12.96 (+/−3.98) | >3000* |
| PB1₁₋₁₅ A_{T6Y, L7F} | ..... [symbol] ........ | 7.51 (+/−0.71) | 345.0 (+/−81.5) |
| PB1₁₋₁₅ A_{L7F} | ...... [symbol] ........ | >3000* | >3000* |
| PB1₁₋₁₅ A_{T5Y} | ..... [symbol] ......... | 21.64 (+/−1.48) | 107.1 (+/−31.3) |
| PB1₁₋₁₅ A_{T5F} | .....F......... | 2.84 (+/−0.48) | 750.4 (+/−249.6) |
| PB1₁₋₁₅ A_{T6W} | .....W......... | 3.40 (+/−0.51) | 628.3 (+/−389.1) |
| PB1₁₋₁₅ A_{T6H} | .....H......... | 292.16 (+/−34.04) | >3000* |
| PB1₁₋₁₅ A_{T5C} | .....C......... | 43.58 (+/−5.67) | >3000* |

*highest concentration of competitive peptide used

A comprehensive and qualitative overview on further peptides with high inhibitory activity is provided in Table 1b. In the table the amino acid sequences at positions X₅ to X₁₀ of wild type A mutants are indicated.

TABLE 1b
qualitative overview of further preferred peptides

Amino acid Position

| | X₅ | X₆ | X₇ | X₈ | X₉ | X₁₀ |
|---|---|---|---|---|---|---|
| Wild type A | P | T | L | L | F | L |
| | | I | | I | Y | |
| | | L | | | W | |
| | | V | | | | |

In Table 1c the amino acid sequences at amino acid residues X₅ to X₁₀ of wild type A mutants are indicated. Said peptides exhibit lower activities than the above mentioned peptides according to Tables 1a and 1b.

TABLE 1c
qualitative overview of further peptides

Amino acid Position

| | X₅ | X₆ | X₇ | X₈ | X₉ | X₁₀ |
|---|---|---|---|---|---|---|
| Wild type A | P | T | L | L | F | L |
| | | M | | F | H | I |
| | | A | | M | L | |
| | | | | | R | |
| | | | | | S | |

Based on the above presented information and results, it is clear for the person skilled in the art, that the synthesized or isolated influenza virus replication-inhibiting peptides interacting with the inhibition target for the small molecules compounds according to the invention comprise an amino acid sequence of X₅X₆X₇X₈X₉X₁₀, wherein X₅ is P; X₆ is T, Y, F, W, H, C, I, L, V, A or M; X₇ is L or F; X₈ is L, I, F or M; X₉ is F, Y, W, H, L, R or S, and X₁₀ is L, I or Y. Said amino acid sequence is at least 60%, preferably at least 70%, more preferably at least 80% or 90% identical to the polypeptide according to the wild type PB1₁₋₁₁A which is MDVNPTLLFLK. Within the aforementioned group of peptides, those peptides are preferred which comprising the amino acid sequence of X₆X₇X₈X₉X₁₀, wherein X₆ is T, Y, F, W, H, C, I, L or V; X₇ is L or F; X₈ is L or I; X₉ is F, Y or W and X₁₀ is L. Even more preferred according to certain embodiments are peptides that comprise the amino acid sequence of X₆X₇, wherein X₆ is T, Y, F, W, H, C, I, L or V and X₇ is L or F.

Effective peptides advantageously comprise at least 11 residues X₁₋₁₁, whereby preferably the proteins comprise the amino acid sequence MDVNPX6X7LFLKVPAQ wherein X6 is selected from the group: T, Y, F, W, H. C, A, I, L, V or M and X7 is selected from the group L or F. A preferred peptide comprises an amino acid sequence elected from the group: MDVNPYFLFLKVPAQ, MDVNPYLLFLKVPAQ, MDVNPWLLFLKVPAQ or MDVNPFLLFLKVPAQ. According to further preferred embodiments the peptides comprise at least 15 residues X₁₋₁₅ according to the wild type PB1₁₋₁₅A but not the wild type sequence MDVNPTLLFLKVPAQ.

Table 2 shows the 50%-inhibitory concentrations (IC₅₀) of FluA-derived PB1 peptides determined by competitive ELISA. Peptide PB1₁₋₂₅A was immobilized on microwell plates and incubated with increasing concentrations of competitor peptides and cell extract containing HA-tagged PA of FluA. Bound PA was detected by HA-specific antibodies as described above. Standard deviation is shown in parenthesis. Asterisks indicate highest concentrations of peptides used without detectable inhibitory effect. Grey boxes highlight amino acids that are part of the 3₁₀-helix, which comprises the core PA-binding region of PB1. Amino acids known to form hydrogen bonds with PA residues are represented in bold. The systematic truncation of the 25 mer peptide comprising the PA-binding domain of PB1 at the N- and C-terminus showed—based on the ELISA assay results—that i) the 25 mer peptide can be truncated at the C-terminus until the first 14 or even 13 N-terminal amino acids remain without losing ability to inhibit the bound peptide-PA interaction. Truncation at the C-terminus down to the first 12 or even 11 amino acids resulted in peptides which still showed considerable activity. The systematic truncation showed further that ii) N-terminal truncation is not possible without major loss in inhibitory activity of the peptide.

TABLE 2
Inhibitory concentrations (IC₅₀) of FluA-derived PB1 peptides

| FluA-PB1 peptides (aa) | | IC₅₀ (nM) |
|---|---|---|
| 3₁₀-helix | | |
| 1-25 3-25 5-25 7-25 9-25 11-25 | 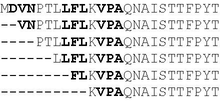 | 1.80 (+/− 0.49) 661.77 (+/− 22.08) 483.20 (+/− 51.98) >3000* >3000* >3000* |
| 1-20 1-18 1-16 1-15 1-14 1-13 1-12 1-11 1-10 1-9 1-8 1-7 1-6 | 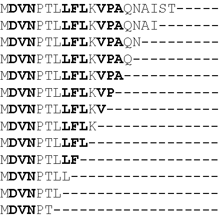 | 33.80 (+/− 5.53) 29.45 (+/− 5.16) 45.86 (+/− 4.22) 43.32 (+/− 5.31) 34.53 (+/− 2.19) 138.17 (+/− 7.88) 643.93 (+/− 180.75) 899.53 (+/− 54.31) >3000* >3000* >3000* >3000* >3000* |

*highest concentration of competitive peptide used

Table 3 illustrates the inhibitory concentrations (IC₅₀) of FluA-derived competitor peptides determined by ELISA. Peptide PB1₁₋₂₅A was again immobilized on microwell plates and incubated with increasing concentrations of competitor peptide and cell extract containing HA-tagged PA of FluA. HA-specific antibodies detected bound PA. Standard deviations are shown in parenthesis. Asterisks indicate highest concentrations of peptides used without detectable inhibitory effect.

TABLE 3
Inhibitory concentrations (IC₅₀)
of FluA-derived PB1 peptides

| Competitive | | |
|---|---|---|
| peptide | | IC50 in nM |
| PB1₁₋₁₅ A | MDVNPTLLFLKVPAQ | 43.32 (+/−5.31) |
| PB1₁₋₁₅ A M1A | [symbol] DVNPTLLFLKVPAQ | 460.30 (+/−27.85) |
| PB1₁₋₁₅ A D2A | M [symbol] VNPTLLFLKVPAQ | 209.17 (+/−44.62) |
| PB1₁₋₁₅ A V3A | MD [symbol] NPTLLFLKVPAQ | 154.93 (+/−18.18) |
| PB1₁₋₁₅ A N4A | MDV [symbol] OTKKFKJVOAQ | >3000* |
| PB1₁₋₁₅ A P5A | MDVN [symbol] TLLFLKVPAQ | 2728.67 (+/−133.43) |
| PB1₁₋₁₅ A T6A | MDVNP [symbol] LLFLKVPAQ | 701.87 (+/−20.59) |
| PB1₁₋₁₅ A L7A | MDVNPT [symbol] LFLKVPAQ | >3000* |
| PB1₁₋₁₅ A L8A | MDVNPTL [symbol] FLKVPAQ | >3000* |
| PB1₁₋₁₅ A F9A | MDVNPTLL [symbol] LKVPAQ | >3000* |
| PB1₁₋₁₅ A L10A | MDVNPTLLF [symbol] KVPAQ | >3000* |
| PB1₁₋₁₅ A K11A | MDVNPTLLFL [symbol] VPAQ | 1290.33 (+/−210.37) |
| PB1₁₋₁₅ A V12A | MDVNPTLLFLK [symbol] PAQ | 707.87 (+/−168.54) |
| PB1₁₋₁₅ A P13A | MDVNPTLLFLKV [symbol] AQ | 257.93 (+/−36.76) |
| PB1₁₋₁₅ A M1D | [symbol] DVNPTLLFLKVPAQ | 1375.67 (+/−268.11) |
| PB1₁₋₁₅ A V3D | MD [symbol] NPTLLFLKVPAQ | >3000* |
| PB1₁₋₁₅ A N4D | MDV [symbol] PTLLFLKVPAQ | >3000* |
| PB1₁₋₁₅ A P5D | MDVN [symbol] TLLFLKVPAQ | >3000* |
| PB1₁₋₁₅ A T6D | MDVNP [symbol] LLFLKVPAQ | 2067.67 (+/−584.98) |
| PB1₁₋₁₅ A L7D | MDVNPT [symbol] LFLKVPAQ | >3000* |
| PB1₁₋₁₅ A L8D | MDVNPTL [symbol] FLKVPAQ | >3000* |
| PB1₁₋₁₅ A F9D | MDVNPTLL [symbol] LKVPAQ | >3000* |
| PB1₁₋₁₅ A L10D | MDVNPTLLF [symbol] KVPAQ | >3000* |
| PB1₁₋₁₅ A K11D | MDVNPTLLFL [symbol] VPAQ | >3000* |
| PB1₁₋₁₅ A V12D | MDVNPTLLFLK [symbol] PAQ | 2302.67 (+/−280.39) |
| PB1₁₋₁₅ A P13D | MDVNPTLLFLKV [symbol] AQ | 1097.47 (+/−217.54) |

FIG. 1 shows binding and inhibitory activity of PB11-25AT6Y. Based on FIG. 1 the binding and inhibitory activity of peptides binding to the inhibition target with a focus on the preferred protein PB1₁₋₂₅A_{T6Y} shall be illustrated in the following part of the description. FIG. 1a shows in the upper panel the alignment of the consensus sequence of the N-terminal 25 amino acids of FluA and FluB PB1, wherein the dotted box indicates the 3₁₀-helix comprising the core PA-binding domain of PB1 and the FluA-specific and FluB-specific amino acids are printed in bold letters. Middle and lower panels show the alignment of the N-terminal 25 amino acids of all available FluA and FluB sequences derived from PB1 full length sequences provided by the NCBI influenza virus database.

The binding of HA-tagged PA subunits from cell extracts to the immobilized peptides corresponding to the domains of FluA PB1 (PB1₁₋₂₅A), FluB PB1 (PB1₁₋₂₅B) or FluA PB1 T6Y (PB1₁₋₂₅A_{T6Y}) determined by ELISA is shown in FIG. 1b. Signals using the cognate peptide and lysate were normalized to 1. Binding of the PA subunits to the control peptides was not observed. Upper panels: Western blot of the PA-containing cell extracts used. Molecular weights shown in kilodaltons.

FIG. 1c provides graphic information on the structure of FluA PB1₁₋₁₅ bound to FluA PA. T6 forms a hydrogen bond to a water molecule. Molecular modeling suggests that the aromatic side chain in the mutant T6Y fits into a hydrophobic pocket and displaces the water molecule. The polymerase inhibitory activity of PB1₁₋₂₅-derived GFP fusion proteins in FluA and FluB polymerase reconstitution assays is shown in FIG. 1d. The activity in experiments containing all viral plasmids and Flag-GFP was set to 100%.

FIG. 1e shows a plaque reduction assay using PB1₁₋₂₅A-Tat; PB1₁₋₂₅A_{T6Y}-Tat; PX-Tat (control peptide) with FluA, FluB and VSV (vesicular stomatitis virus). A H₂O control was used to standardize the assay to 100%. Note that PB1₁₋₂₅B-Tat could not be tested due to insolubility. Error bars represent standard deviations.

Virus type-specific interaction of PA with PB1 is illustrated in FIG. 2. FIG. 2a shows A/SC35M- and B/Yamagata/73-derived PB1 chimeras used in tests according to FIG. 2b. Note that all PB1 proteins were expressed with C-terminal HA-tags. FIG. 2b shows human 293T cells which were transfected with expression plasmids coding for the indicated PB1 proteins and the C-terminally hexahistidine-tagged PA of FluA (FluAPA_{His}). Cell lysates were prepared 24 hours post transfection and subjected to immunoprecipitation (IP) using anti-HA (aHA) agarose. Precipitated material was separated by SDS-PAGE and analyzed by Western blot for the presence of either His- or HA-tagged polymerase subunits using appropriate antibodies. Protein expression was controlled by analyzing equal amounts of cell lysate. Molecular weights are shown in kilodaltons. The 25-mer peptide, PB1₁₋₂₅A, comprising a helical domain inhibits the polymerase activity and replication of FluA, whereas the activity of FluB polymerase is not affected.

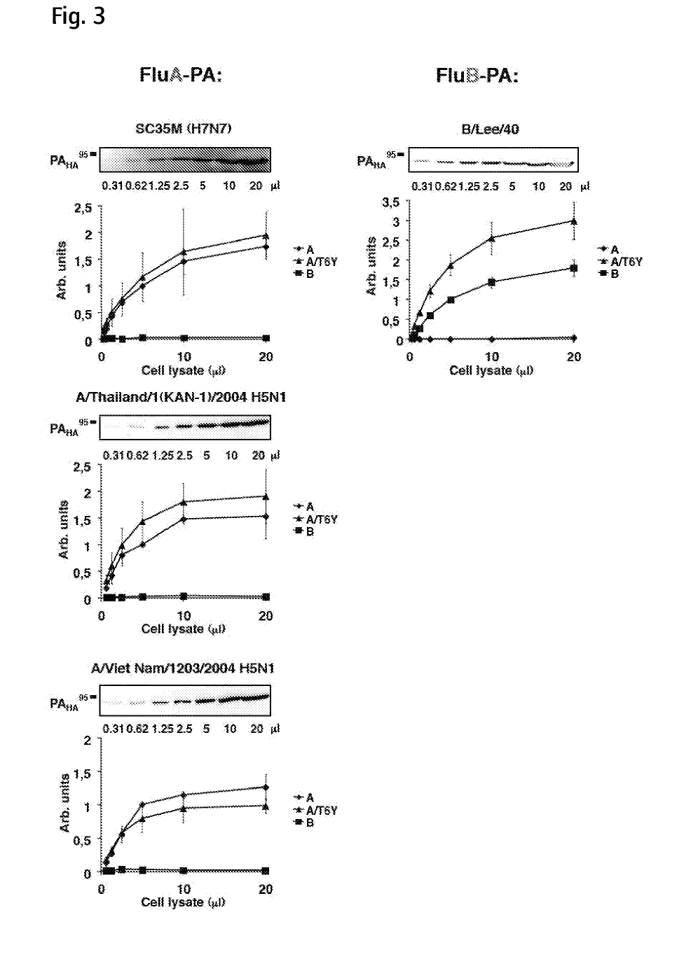

In FIG. 3 dual-binding properties of the FluA/B peptide chimera PB1₁₋₂₅A_{T6Y} are illustrated in comparison to PB1₁₋₂₅A and PB1₁₋₂₅B. The Lower panels show peptides PB1₁₋₂₅A, PB1₁₋₂₅B or PB1₁₋₂₅A_{T6Y} immobilized on microwell plates and incubated with increasing concentrations of cell extract containing the indicated PA-HA from FluA or FluB strains. Bound PA-HA was detected by HA-specific antibodies and peroxidase-labeled secondary antibodies. Binding efficiency was quantified by measuring substrate conversion at 405 nm. Standard deviations are indicated by error bars. Experiments were repeated in triplicates. Upper panels show analysis of corresponding amounts of cell lysate by Western blot controlled protein expression. Molecular weights are shown in kilodaltons.

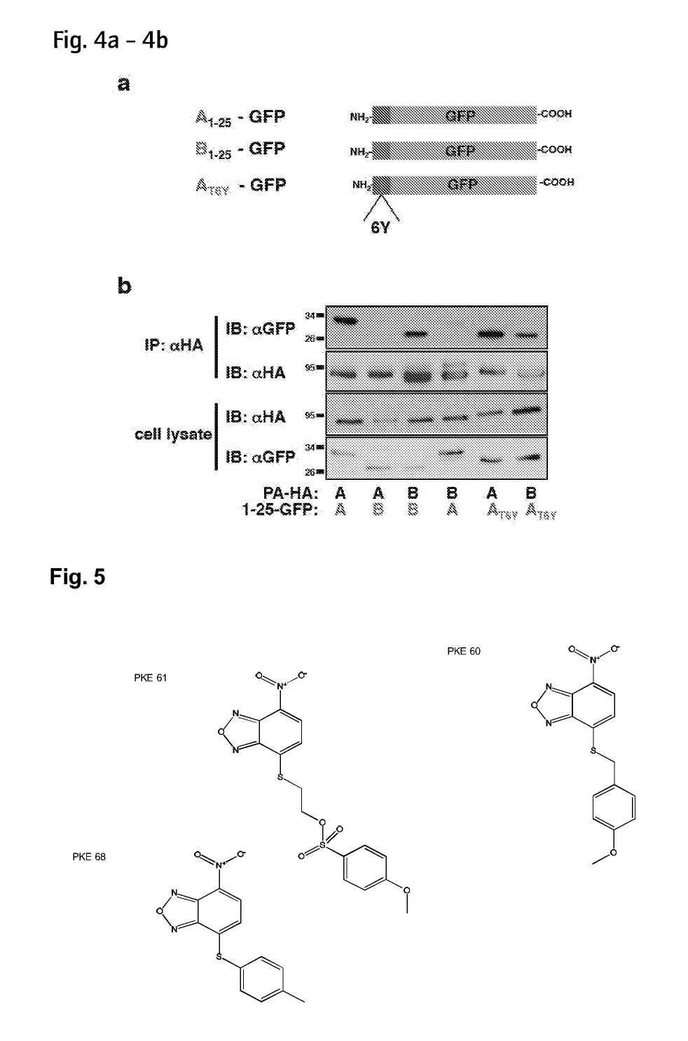

FIG. 4a shows GFP-PB1 fusion proteins used in tests according to FIG. 4b. The complex formation of PB1₁₋₂₅-derived GFP fusion proteins and HA-tagged PA of FluA and FluB is shown in FIG. 4b. Indicated proteins were expressed in human 293T cells and binding of the GFP fusion proteins was analyzed by immunoprecitation (IP) of PA using anti-HA agarose and subsequent immunoblotting (IB). Precipitated material was analyzed by Western blot using the indicated antibodies for the presence of either HA-tagged PA or GFP. Molecular weights are shown in kilodaltons.

### Influenza Type A and B: Materials and Methods

Virus strains: For the infection experiments A/WSN/33 (H1N1) according to Ghanem et al. (2007) and A/Thailand/1(Kan-1)/2004 according to Chockephaibulkit et al. (2005), B/Yamagat/73 according to Norton (1987) and VSV (serotype Indiana) as described in Schwemmle (1995) were used.

Plasmid constructions: Plasmids pCA-Flag-GFP and pCA-PB1₁₋₂₅A-GFP, pCA-PB1-HA, the FluA minireplicon plasmids and the expression plasmids for the FluB minireplicon are described in Ghanem (2007), Mayer (2007) and Pleschka (1996). The FluB minigenome expression plasmid, pPoll-lucRT_B, was obtained by cloning the firefly luciferase ORF (inverse orientation) flanked by the non-coding region of the segment 8 of the B/Yamagata/73 into the Sapl-digested plasmid pPoll-Sapl-Rib according to Pleschka (1996). For the construction of pCA-PB1₁₋₂₅B-GFP, a linker containing the first 25 codons of PB1 (B/Yamagata/73) was cloned into the EcoRI/NotI sites of pCA-Flag-GFP plasmid, replacing the Flag-coding sequence with PB1₁₋₂₅B. Site directed mutagenesis was carried out with pCA-PB1₁₋₂₅A-GFP to create the plasmid pCA-PB1₁₋₂₅A_{T6Y}-GFP. The ORFs of PB1 (B/Yamagata/73) and PA (A/SC35M, A/Thailand/1(KAN-1)/04, A/Vietnam/1203/04, B/Yamagata/73, B/Lee/40) were PCR amplified with sense primers containing an NotI site (FluA strains) or a EcoRI site (FluB strains) upstream of the initiation codon and antisense primers with a deleted stop codon followed by an Xmal site, a coding sequence for an HA-tag and a XhoI site. The PCR products were cloned into a modified pCAGGsvector (Schneider, 2003) digested either with EcoRI/XhoI or NotI/XhoI, resulting in pCA-PB1-HA or pCA-PA-HA plasmids, coding for C-terminal tagged versions of the polymerase subunits. To obtain the pCA-PA_{A/SC35M}-His plasmid, pCA-PA_{A/SC35M}-HA was digested with Xmal/XhoI and the HA coding sequence was replaced by a 6×His-linker. The A/B-chimeric expression plasmids were obtained by assembly PCR using the pCAPB1-HA plasmids of SC35M and B/Yamagata/73 and by cloning the resulting PCR product in pCA-PB1_{B/Yamagata/73}-HA digested with EcoRI/EcoRV.

Reconstitution of the influenza virus polymerase activity: HEK293T cells were transiently transfected with a plasmid mixture containing either FluA- or FluB-derived PB1-, PB2-, PA- and NP-expression plasmids, polymerase I (Pol I)-driven plasmid transcribing an influenza A or influenza B virus-like RNA coding for the reporter protein firefly luciferase to monitor viral polymerase activity and with expression plasmids coding for the indicated GFP fusion proteins. Both minigenome RNAs were flanked by non-coding sequences of segment 8 of FluA and FluB, respectively. The transfection mixture also contained a plasmid constitutively expressing Renilla luciferase, which served to normalize variation in transfection efficiency. The reporter activity was determined 24 h post transfection and normalized using the Dual-Glu® Lufierase Assay System (Promega). The activity observed with transfection reactions containing Flag-GFP were set to 100%.

Peptide synthesis: The solid-phase synthesis of the peptides was carried out on a Pioneer automatic peptide synthesizer (Applied Biosystems, Foster City, USA) employing Fmoc chemistry with TBTU/diisopropylethyl amine activation. Side chain protections were as follows: Asp, Glu, Ser, Thr and Tyr: t-Bu; Asn, Gln and His: Trt; Arg: Pbf; Lys and Trp: Boc. Coupling time was 1 h. Double couplings were carried out if a difficult coupling was expected according to the program Peptide Companion (Coshi-Soft/PeptiSearch, Tucson, USA). All peptides were generated as carboxyl amides by synthesis on Rapp S RAM resin (Rapp Polymere, Tubingen, Germany).Biotin was incorporated at the C-terminus of indicated peptides with Fmoc-Lys(Biotin)-OH (NovaBiochem/Merck, Nottingham, UK) and TBTU/diisopropylethylamine activation for 18 h, followed by coupling of Fmoc-β-Ala-OH for 1 h. Peptides were cleaved from the resin and deprotected by a 3 h treatment with TFA containing 3% triisobutylsilane and 2% water (10 ml/g resin). After precipitation with t-butylmethylether, the resulting crude peptides were purified by preparative HPLC(RP-18) with water/acetonitrile gradients containing 0.1% TFA and characterized by analytical HPLC and MALDI-MS. Some peptides were synthesized by peptides&elephants (Nuthetal, Germany) and subsequently purified and characterized as described above.

Immunoprecipitation experiments: HEK293T cells were transfected with the indicated plasmids in 6-well plates using Metafectene (Biontex, Martinsried, Germany). Cells were incubated 24 h post transfection with lysis buffer (20 mM Tris pH7.5, 100 mM NaCl, 0.5 mM EDTA, 0.5% NP-40, 1% Protease inhibitor Mix G, (Serva, Heidelberg, Germany), 1 mM DTT) for 15 min on ice. After centrifugation by 13.000 rpm at 4° C. supernatant was incubated with anti HA-specific antibodies coupled to agarose beads (Sigma) for 1 h at 4° C. After three washes with 1 ml of washing buffer (lysis buffer without protease inhibitor mix), bound material was eluted under denaturing conditions and separated on SDSPAGE gels and transferred to PVDF membranes. Viral polymerase subunits and GFP fusion proteins were detected with antibodies directed against the HA-tag (Covance, Berkeley, Calif.) or His-tag (Qiagen) or GFP-tag (Santa Cruz Biotechnology).

Plaque reduction assay: The experiments were carried out as described by Schmidke (2001) with modifications. Confluent MDCK cells were infected with 100 PFU of A/WSN/33, B/Yamagata/73, A/KAN-1, or VSV/Indiana in PBS containing BSA at room temperature. After removal of the inoculum, cells were overlaid with medium (DMEM with 20 mM Hepes, 0.01% DEAE Dextran, 0.001% NaHCO₃) containing 1% Oxoidagar and candidate peptides or small molecule compounds at the indicated concentrations. After incubation for 24 h (VSV), 48 h (A/WSN/33, A/KAN-1) at 37° C. with 5% CO₂, or 72 h at 33° C. with 5% CO₂ (B/Yamagata/73) respectively, cells were fixed with formaldehyde and stained with crystal violet. Plaques were counted and mean plaque number of the water control was set to 100%.

Enzyme-Linked ImmunoSorbent Assay (ELISA): For the ELISA microwell plates (Pierce) were incubated with saturating concentrations of peptides at room temperature, washed and subsequently incubated at room temperature with HA-tagged PA. To obtain PA-HA, 293T cells were seeded into 94 mm-dishes, transfected with the respective plasmid and treated with lysis buffer 24 h post transfection as described in detail by Mayer et al. (2007). After washing the microwell plates, the wells were incubated with an HA-specific primary antibody (Covance), followed by three washes and an incubation with a peroxidase-coupled secondary antibody (Jackson Immuno Research, Newmarket, UK) for further 30 min. After the final wash step, ABTS-substrate (Sigma, ready-to-use solution) was added and the optical density was determined at 405 nm.

The competition ELISA was carried out as described above with the exception that the candidate peptide or small molecule competitor compound were added to wells of the plate with bound peptides prior to addition of the cell extract containing HA-tagged PA subunits.

Fluorescence Polarization (FP) Assay: The test sample includes a known binding pair of proteins or protein subunits including a fluorescent label, which can be analyzed according to a preferred embodiment of the present invention by fluorescence polarization. Here, we use the interaction of Influenza A virus polymerase subunit PB1, represented by the first 25, N-terminal amino acids, and subunit PA. The test sample is then contacted with a candidate peptide or small molecule inhibitor compound and the resulting fluorescence polarization is determined. The ability of the compound to cause dissociation of or otherwise interfere with or prevent binding of the proteins or protein subunits is monitored by fluorescence polarization (FP). FP measurements allow for discrimination between fluorescently labeled bound and unbound proteins, peptides, subunits or fragments thereof. The FP of the fluorescently labeled first fragment rotates rapidly in solution and, therefore, has randomized photo-selected distributions, which result in the small observed FP. When the fluorescently labeled first fragment of the first subunit interacts with the fragment of the second subunit, which is typically a larger, more slowly rotating molecule, the rotation of the fluorescently labeled first fragment slows and the fluorescence polarization increases. Accordingly, disruption of the subunit interaction by a test compound provides a decrease in the fluorescence polarization, which is indicative of inhibition of the protein interactions. The FP measurements in the presence of a test compound can be compared with the FP measurements in the absence of the test compound. Comparison can be made manually by the operator or automatically by a computer, especially in high throughput assays using 384-well plates.

For protein purification influenza A virus polymerase subunit PA was cloned into a suitable expression vector with a C-terminally attached 6×His-linker or hemagglutinine epitope (HA). Human 293T cells were transfected with the plasmid. Cell lysates were prepared 24 hours post transfection using lysis buffer (20 mM TrisHCl pH 7.5, 100 mM NaCl, 0.5 mM EDTA, 0.5% NP-40, 1 mM DTT and 1% Protase inhibitor mix) For purification from the lysate, PA subunit was bound to Ni- or anti-HA-agarose and washed with lysis buffer without protease mix. After elution with HA-peptide in 20 mM TrisHCl pH 7.5, 150 mM NaCl, 0.5 mM EDTA, 1 mM DTT and 5% Glycerol, PA-protein was concentrated when necessary using Vivaspin20 50K columns and frozen at −80° C. until further use. After thawing, the elution buffer was exchanged to low fluorescent grade reagents and any HA-peptide was removed simultaneously using 10-DG Bio-Gel columns.

Fluorescently labeled peptide corresponding to the 25 first N-terminal amino acids of Influenza A virus polymerase subunit PB1 at 3 nM concentration was added to 10 μM HA-PA in 20 mM TrisHCl pH 7.5, 150 mM NaCl, 0.5 mM EDTA, 1 mM DTT, 5% Glycerol and 100 mg/ml bovine gamma globulin. The mix was distributed into black 384-well plates to a total volume of 20 μl per well and kept on ice. Test compounds solved in DMSO were added to a final concentration of 25 μM. After incubation for 10 minutes at room temperature, plates were read using an Infinite F200 reader (Tecan). FP values of the wells containing test compounds were compared to wells without test compounds, without DMSO and with peptide only.

Sequence alignment: Alignments were performed with MUSCLE as described in Edgar (2004) using the full-length sequences provided from the public influenza virus database (http://www.ncbi.nlm.nih.gov/genomes/FLU/FLU.html).

Modelling: Manual docking of the mutated peptide into the PA(C)—PB1(N) crystal structure (He et al., 2008) and subsequent minimization was performed with Accelrys Discovery Studio.

### Respiratory Syncytial Virus: Materials and Methods

Activity of compounds in reducing RSV induced cell death: HEp-2 cells (obtained from ATCC) were seeded in 96-well plates (1.5×10⁴ cells per well) and grown in MEM-alpha medium containing 10% FBS (Gibco-BRL) for 24 h. To infect cells, 500 pfu of RSV Long strain (obtained from ATCC) were added in 50 μl of OptiMEM (Gibco-BRL) for 1 h. Cells were then incubated in the presence of a serial dilution of compounds (from 100 to 0.14 μM) in MEM-alpha containing 2% FBS for 72 h. Cells were fixed in 3.7% formaldehyde and stained with 0.025% of crystal violet (Sigma). The integrity of the cellular monolayer was measured at 540 nm using a microplate reader. The activity of the compounds to reduce virus-induced cell death is expressed as the mean of three independent experiments each performed in triplicates.

### Experimental Results

Protein-protein interactions (PPIs) are crucial to most, if not all, biological processes. Of the roughly 30,000 protein sequences that comprise the human proteome, only about 1% have been successfully targeted with small-molecule drugs. Yet, most of the conventional targets in drug discovery fall into the same few structural or functional families such as enzymes or G protein-coupled receptors (GPCRs). They typically share the property that the natural substrates or ligands, with which they interact are themselves small organic molecules. Historically there has been notably little success in developing drug-like inhibitors of proteins whose natural ligands are other proteins. Designing a small molecule to bind to a protein-protein interface and inhibit the interaction poses several challenges, including the initial identification of suitable PPIs, the surface area of the interface, and the localization of “hot spots”. Thus, small molecule inhibition of PPIs is a challenging area in drug discovery.

The present invention uses the fact that proteomes of many viruses and PPIs crucial for viral replication are described in the literature. For any proteome of interest, this data is according to the novel method supplemented with proteomic approaches for identification of PPIs like yeast two-hybrid or co-immuno precipitation screening in order to identify potential target regions for development of PPI inhibitors. Subsequently, a unique combination of phylogenetic analysis and structure prediction or structure analysis (where applicable) of the protein partners involved detects druggable protein-binding domains. Within the present disclosure, the term druggable denotes preferably protein-binding domains which can be blocked, altered or modified by small molecules in a way that the protein-protein interaction is inhibited or disrupted. The term small molecules denotes organic molecules, preferably synthetic organic molecules (not peptides), which have a molecular weight below 1500, preferably below 1000 and most preferred below 500 u. It has been found, that these domains bear a couple of characteristic features: (i) helical structure, (ii) hydrophobic character and (iii) high conservation among all virus strains. It has been shown that they tend to be located at a terminal end of the protein or are located on their surface. The peptides corresponding to these potential binding domains are synthesized in an overlapping way and tested for their ability to bind the protein partner involved in the PPI.

If peptides resembling short, (less than 20 amino acids) continuous binding domains are identified, these are used for the development of a binding assay, preferably an ELISA or fluorescence polarization (FP) assay, which is afterwards employed in a high-throughput screening campaign for small molecule and/or peptidic inhibitors of the PPI.

The PPI inhibitors identified by the novel method according to the present invention, as opposed to conventional active site inhibitors, could offer a particular advantage when it comes to antivirals since it should be safe to assume that resistance development occurs at a much slower pace.

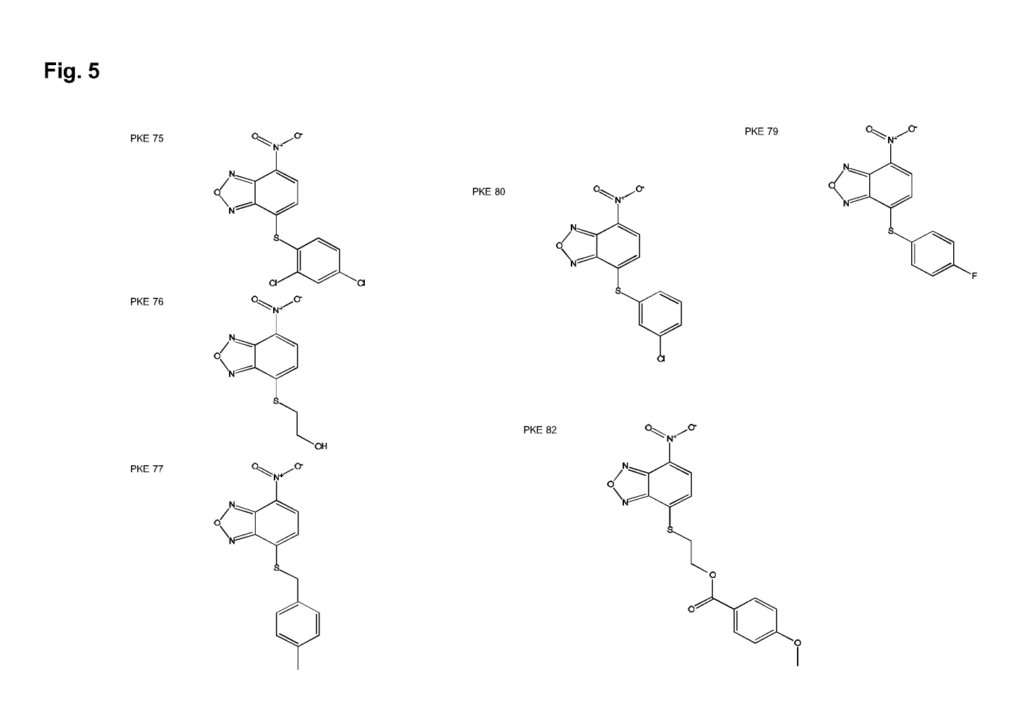

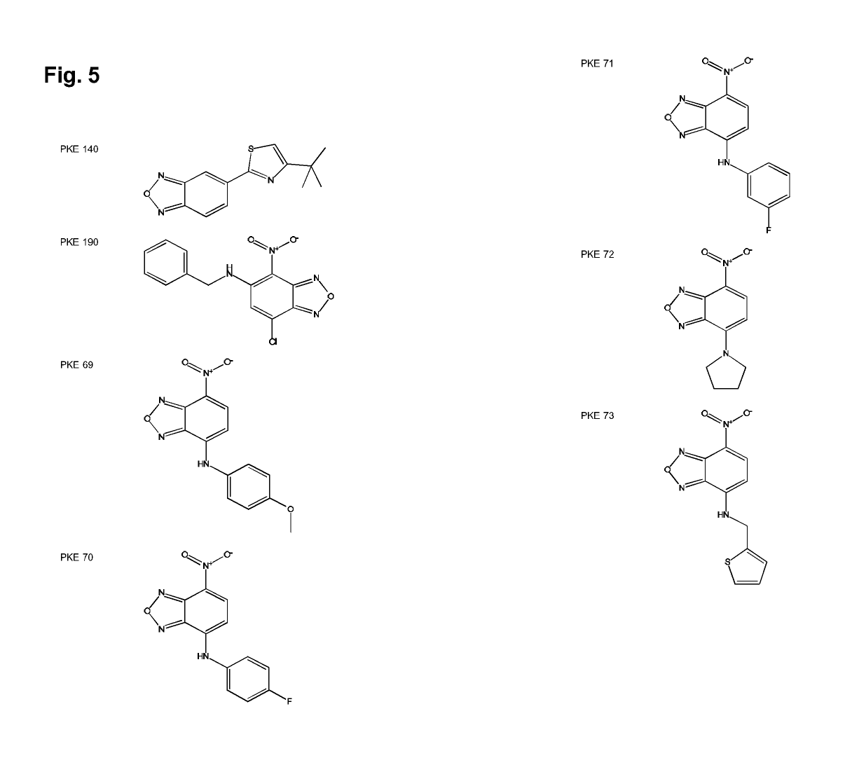

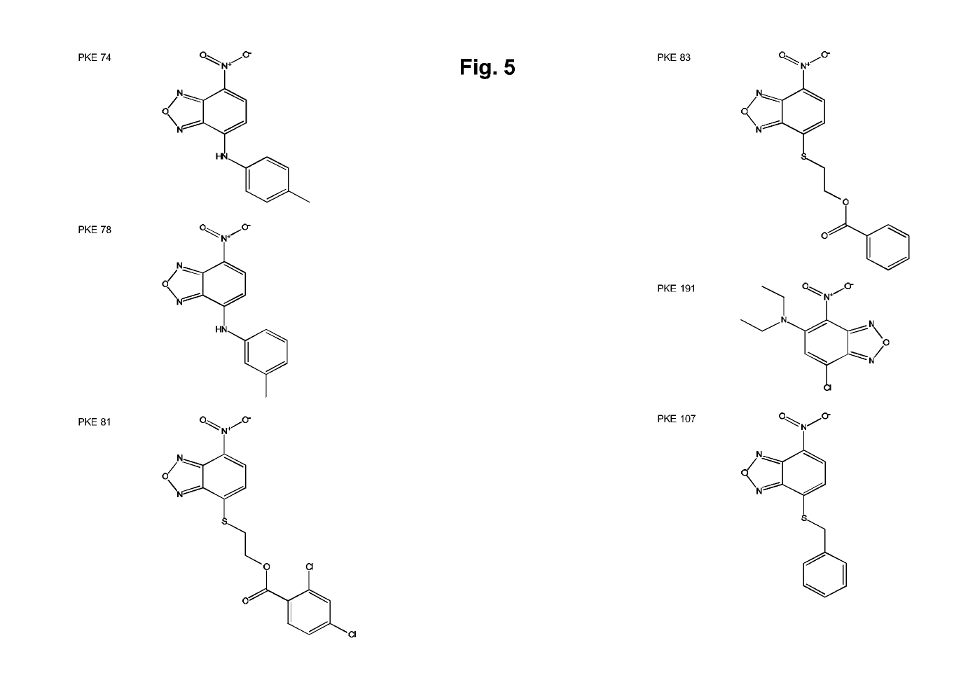

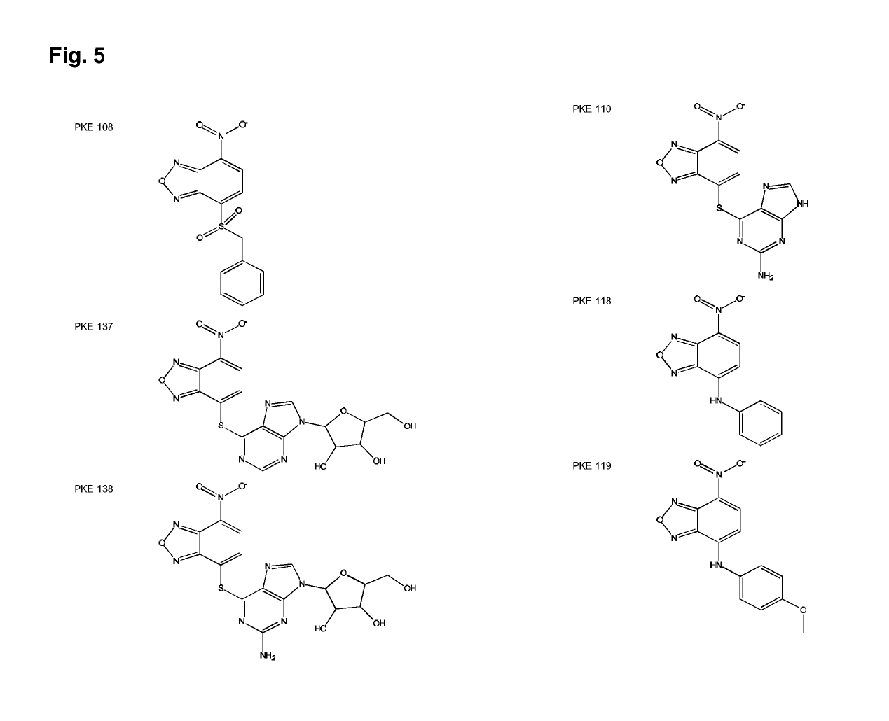

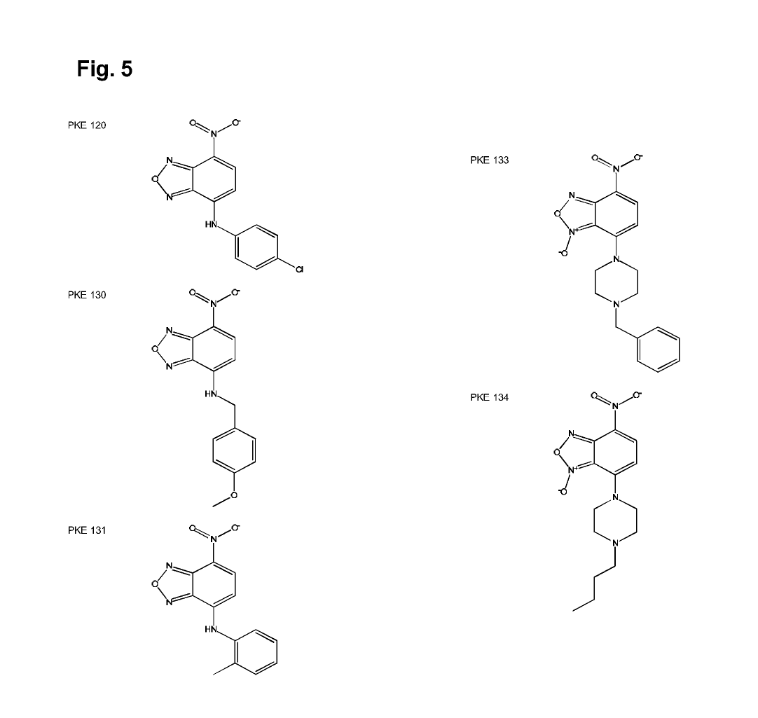

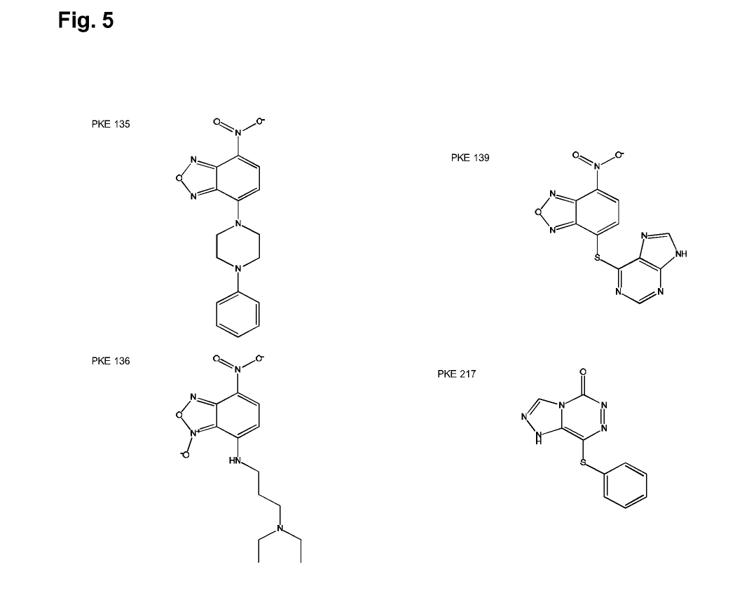

In order to identify chemical compounds that efficiently interrupt or disrupt the interaction between PB1 and PA, for example by binding to the inhibition target on PA of FluA and FluB, the competitive ELISA assay described above for the influenza peptides was repeated with a number of small molecule compounds obtained from corresponding compound libraries from Maybridge Ltd., Cambridge, UK (www.maybridge.com) and from the Developmental Therapeutics Program NCl/NIH (http://www.dtp.nci.nih.gov) of the U.S. National Institutes of Health. The tested compounds are listed in Table 4, together with their systematic name, the source and the product code, and the found activity in the ELISA assay. The corresponding structures are shown in FIG. 5.

TABLE 4
Compounds tested with competitive ELISA

| | | Source*/ | Active in |
|---|---|---|---|
| Comp. ID | Compound name | Product code | ELISA |
| PKE060 | 4-[(4-methoxybenzyl)thio]-7-nitro-2,1,3-benzoxadiazole | M/KM06831 | Yes |
| PKE061 | 2-[(7-nitro-2,1,3-benzoxadiazol-4-yl)thio]ethyl 4- | M/KM06890 | Yes |
| | methoxybenzene-1-sulfonate | | |
| PKE068 | 4-[(4-methylphenyl)thio]-7-nitro-2,1,3-benzoxadiazole | M/KM06815 | Yes |
| PKE069 | N4-(4-methoxyphenyl)-7-nitro-2,1,3-benzoxadiazol-4-amine | M/KM06816 | No |
| PKE070 | N4-(4-fluorophenyl)-7-nitro-2,1,3-benzoxadiazol-4-amine | M/KM06820 | No |
| PKE071 | N4-(3-fluorophenyl)-7-nitro-2,1,3-benzoxadiazol-4-amine | M/KM06822 | No |
| PKE072 | 4-nitro-7-tetrahydro-1H-pyrrol-1-yl-2,1,3-benzoxadiazole | M/KM06824 | No |
| PKE073 | N4-(2-thienylmethyl)-7-nitro-2,1,3-benzoxadiazol-4-amine | M/KM06825 | No |
| PKE074 | N4-(4-methylphenyl)-7-nitro-2,1,3-benzoxadiazol-4-amine | M/KM06826 | No |
| PKE075 | 4-[(2,4-dichlorophenyl)thio]-7-nitro-2,1,3-benzoxadiazole | M/KM06828 | Yes |
| PKE076 | 2-[(7-nitro-2,1,3-benzoxadiazol-4-yl)thio]ethan-1-ol | M/KM06833 | Yes |
| PKE077 | 4-[(4-methylbenzyl)thio]-7-nitro-2,1,3-benzoxadiazole | M/KM06835 | Yes |
| PKE078 | N4-(3-methylphenyl)-7-nitro-2,1,3-benzoxadiazol-4-amine | M/KM06836 | No |
| PKE079 | 4-[(4-fluorophenyl)thio]-7-nitro-2,1,3-benzoxadiazole | M/KM06837 | Yes |
| PKE080 | 4-[(3-chlorophenyl)thio]-7-nitro-2,1,3-benzoxadiazole | M/KM06838 | Yes |
| PKE081 | 2-[(7-nitro-2,1,3-benzoxadiazol-4-yl)thio]ethyl 2,4- | M/KM06863 | No |
| | dichlorobenzoate | | |
| PKE082 | 2-[(7-nitro-2,1,3-benzoxadiazol-4-yl)thio]ethyl-4- | M/KM06867 | Yes |
| | methoxybenzoate | | |
| PKE083 | 2-[(7-nitro-2,1,3-benzoxadiazol-4-yl)thio]ethyl benzoate | M/KM06874 | No |
| PKE107 | 4-nitro-7-(phenylmethylsulfanyl)-2,1,3-benzoxadiazole | N/NSC228147 | Yes |
| PKE108 | 4-nitro-7-(phenylmethylsulfonyl)-2,1,3-benzoxadiazole | N/NSC228148 | Yes |
| PKE110 | 6-[(4-nitro-2,1,3-benzoxadiazol-7-yl)sulfanyl]-7H-purin-2-amine | N/NSC348401 | No |
| PKE118 | 4-nitro-N-phenyl-2,1,3-benzoxadiazol-7-amine | N/NSC611541 | No |
| PKE119 | N-(4-methoxyphenyl)-4-nitro-2,1,3-benzoxadiazol-7-amine | N/NSC611543 | No |
| PKE120 | N-(4-chlorophenyl)-4-nitro-2,1,3-benzoxadiazol-7-amine | N/NSC611544 | No |
| PKE130 | N-[(4-methoxyphenyl)methyl]-4-nitro-2,1,3-benzoxadiazol-7-amine | N/NSC240872 | No |
| PKE131 | N-(2-methylphenyl)-4-nitro-2,1,3-benzoxadiazol-7-amine | N/NSC611542 | No |
| PKE133 | 4-nitro-1-oxido-7-[4-(phenylmethyl)piperazin-1-yl]-2,1,3- | N/NSC228099 | No |
| | benzoxadiazol-1-ium | | |
| PKE134 | 7-(4-butylpiperazin-1-yl)-4-nitro-1-oxido-2,1,3-benzoxadiazol-1-ium | N/NSC228106 | No |
| PKE135 | 4-nitro-7-(4-phenylpiperazin-1-yl)-2,1,3-benzoxadiazole | N/NSC288659 | No |
| PKE136 | N,N-diethyl-N′-(4-nitro-1-oxido-2,1,3-benzoxadiazol-1-ium-7- | N/NSC288662 | No |
| | yl)propane-1,3-diamine | | |
| PKE137 | 2-(hydroxymethyl)-5-[6-[(4-nitro-2,1,3-benzoxadiazol- | N/NSC335994 | Yes |
| | 7-yl)sulfanyl]purin-9-yl]oxolane-3,4-diol | | |
| PKE138 | 2-[2-amino-6-[(4-nitro-2,1,3-benzoxadiazol-7-yl) sulfanyl]purin- | N/NSC348400 | Yes |
| | 9-yl]-5-(hydroxymethyl)oxolane-3,4-diol | | |
| PKE139 | 4-nitro-7-(7H-purin-6-ylsulfanyl)-2,1,3-benzoxadiazole | N/NSC348402 | No |
| PKE140 | 5-[4-(tert-butyl)-1,3-thiazol-2-yl]-2,1,3-benzoxadiazole | M/KM07316 | Yes |
| PKE190 | N-benzyl-4-nitro-2,1,3-benzoxadiazol-5-amine | M/BTB15221 | Yes |
| PKE191 | 7-chloro-N,N-diethyl-4-nitro-2,1,3-benzoxadiazol-5-amine | M/BTB15211 | No |
| PKE217 | 8-phenylsulfanyl-6H-[1,2,4]triazolo[4,3-d][1,2,4]triazin-5-one | N/NSC360189 | No |

*M: Maybridge Ltd., Cambridge, UK; N: Developmental Therapeutics Program NCI/NIH

For the compounds with positive ELISA prescreening the inhibitory concentrations (IC₅₀) have been determined (Table 5), in a plaque reduction assay as described above for the influenza peptide studies or with a competitive ELISA assay as described above for the influenza peptide studies. In cases where the solubility was too low to reach the saturation region, the IC₅₀ value was calculated based on the inhibition on the maximum obtainable concentration. If an IC₅₀ value was not obtained, maximum ELISA inhibition at the highest concentration used (1000 μM) is given.

TABLE 5
Influenza inhibitory concentrations (IC₅₀) of compounds

| | IC₅₀ [μM] | IC₅₀ [μM] | Max. Inhibition |
|---|---|---|---|
| Compound ID | (Plaque Red.) | (ELISA) | (ELISA) at 1000 μM |
| PKE060 | 1.00 | | |
| PKE061 | | >1000 | 37% |
| PKE068 | | >1000 | 46% |
| PKE075 | | >1000 | 35% |
| PKE076 | | >1000 | 44% |
| PKE077 | | >1000 | 33% |
| PKE079 | | 125 | |
| PKE080 | | 60 | |
| PKE082 | | 100 | |
| PKE107 | | 125 | |
| PKE108 | | 500 | |
| PKE137 | | 200 | |
| PKE138 | | 250 | |
| PKE140 | | >1000 | 34% |
| PKE190 | | >1000 | 37% |

The compounds that have been found so far to be effective in binding to PA have a basic structure of 2,1,3,-benzoxadiazole. However, compound PKE060 has an IC₅₀ that is considerably lower than PKE079, PKE080, PKE082, PKE107, PKE108, PKE137, and PKE138. The IC₅₀ of the other compounds is not sufficiently low to be physiologically acceptable.

A similar screening was carried out with the plaque reduction assay as described above, with influenza virus (A/WSN/33) and in addition also with RSV (Long strain) for the above-mentioned compounds. The results of the screening are given in Table 6, with the maximum inhibition obtained and, if determinable, the IC₅₀ value. If in an influenza pre-screening assay (competitive ELISA or other) the compound was found to be inactive or having a too high IC₅₀, (IC₅₀ ELISA>100 μM) the influenza assay was not carried out for efficiency reasons.

The maximum inhibition of Influenza A & B activity was only determined for compounds PKE 060 and PKE 080. For compound PKE 060 the maximum inhibition was found to be 75% at 10 μM, and compound PKE 80 was found to be inactive in this assay.

TABLE 6
Influenza and RSV inhibition of compounds

RSV activity

| Compound ID | Maximum inhibition | IC₅₀ [μM] |
|---|---|---|
| PKE060 | inactive | |
| PKE061 | inactive | |
| PKE068 | 24% at 33 μM | |
| PKE069 | inactive | |
| PKE070 | 75% at 11 μM | 4 |
| PKE071 | 73% at 11 μM | 4 |
| PKE072 | 44% at 33 μM | |
| PKE073 | 42% at 33 μM | |
| PKE074 | inactive | |
| PKE075 | 33% at 33 μM | |
| PKE076 | inactive | |
| PKE077 | inactive | |
| PKE078 | 56% at 11 μM | 6 |
| PKE079 | 53% at 11 μM | 7 |
| PKE080 | 54% at 11 μM | 9 |
| PKE081 | 26% at 11 μM | |
| PKE082 | inactive | |
| PKE083 | inactive | |
| PKE107 | inactive | |
| PKE108 | inactive | |
| PKE110 | inactive | |
| PKE118 | 44% at 11 μM | |
| PKE119 | 21% at 11 μM | |
| PKE120 | 67% at 3.7 μM | 2 |
| PKE130 | 48% at 33 μM | |
| PKE131 | 48% at 11 μM | |
| PKE133 | inactive | |
| PKE134 | inactive | |
| PKE135 | inactive | |
| PKE136 | inactive | |
| PKE137 | inactive | |
| PKE138 | inactive | |
| PKE139 | inactive | |
| PKE140 | inactive | |
| PKE190 | inactive | |
| PKE191 | 20% at 11 μM | |

The assessed class of 2,1,3,-benzoxadiazole based compounds seems to be effective in the inhibition of replication of certain virus types. A number of compounds effectively inhibited the replication of influenza virus, particularly influenza A, namely compounds PKE 060, PKE 079, PKE 080, PKE 082, PKE 107, PKE 108, PKE 137, and PKE 138. Surprisingly it was found that a number of other compounds are also effective in the inhibition of RSV replication, namely compounds PKE 068, PKE 070, PKE 071, PKE 072, PKE 073, PKE 075, PKE 078, PKE 079, PKE 080, PKE 081, PKE 118, PKE 119, PKE 120, PKE 130, PKE 131, and PKE 191. A number of compounds inhibits the replication of both virus types.

Without being bound to any theory, it seems that the compounds according to the invention can be very effective broad band inhibitors of virus replication, and thus are a valuable source of effective new medicaments against certain types of the orthomyxoviridae and paramyxoviridae families.

The above-described embodiments of the present invention are intended to be examples only. Alterations, modifications and variations may be effected to the particular embodiments by those of skill in the art without departing from the scope of the invention, which is defined solely by the claims appended hereto. All references are herein incorporated by reference.

### REFERENCES

1. http://www.who.int/csr/disease/influenza/vaccinerecommendationsl/en/.
2. Moscona, A. N Engl J Med 353, 1363-1373 (2005).
3. Davies, W. L. et al. Science 144, 862-863 (1964).
4. Moscona, A. N Engl J Med 360, 953-956 (2009).
5. Dharan, N. J. et al. JAMA 301, 1034-1041 (2009).
6. Pilger, B. D., Cui, C. & Coen, D. M. Chem Biol 11, 647-654 (2004).
7. Brownlee, G. G. & Sharps, J. L. J Virol 76, 7103-7113 (2002).
8. Perales, B. & Ortin, J. J Virol 71, 1381-1385 (1997).
9. Fodor, E. et al. J Virol 76, 8989-9001 (2002).
10. He, X. et al. Nature 454, 1123-1126 (2008).
11. Obayashi, E. et al. Nature 454, 1127-1131 (2008).
12. Ghanem, A. et al. J Virol 81, 7801-7804 (2007).
13. Dostmann, W. R. et al. Proc Natl Acad Sci USA 97, 14772-14777 (2000).
14. Chokephaibulkit, K. et al. Pediatr Infect Dis J 24, 162-166 (2005).
15. Norton, G. P. et al. Virology 156, 204-213 (1987).
16. Schwemmle, M. et al., P. Virology 206, 545-554 (1995).
17. Mayer, D. et al. J Proteome Res 6, 672-682 (2007).
18. Pleschka, S. et al. J Virol 70, 4188-4192 (1996).
19. Schneider, U. et al., M. J Virol 77, 11781-11789 (2003).
20. Schmidtke, M., et al., J Virol Methods 95, 133-143 (2001).
21. Edgar, R. C. Nucleic Acids Res 32, 1792-1797 (2004).

1-7. (canceled)

8. A method for the treatment and/or prevention of a disorder in a subject, wherein the disorder is selected from the group consisting of: influenza type A infection; influenza type B infection; and respiratory syncytial virus infection, the method comprising administering to the subject a therapeutically-effective amount of a 2,1,3-benzoxadiazole compound.

9. The method of claim 8, wherein the 2,1,3-benzoxadiazole compound is selected from the group consisting of:
4-[(4-methoxybenzyl)thio]-7-nitro-2,1,3-benzoxadiazole;
2-[(7-nitro-2,1,3-benzoxadiazol-4-yl)thio]ethyl 4-methoxybenzene-1-sulfonate;
4-[(4-methylphenyl)thio]-7-nitro-2,1,3-benzoxadiazole;
4-[(2,4-dichlorophenyl)thio]-7-nitro-2,1,3-benzoxadiazole;
2-[(7-nitro-2,1,3-benzoxadiazol-4-yl)thio]ethan-1-ol;
4-[(4-methylbenzyl)thio]-7-nitro-2,1,3-benzoxadiazole;
4-[(4-fluorophenyl)thio]-7-nitro-2,1,3-benzoxadiazole;
4-[(3-chlorophenyl)thio]-7-nitro-2,1,3-benzoxadiazole;
2-[(7-nitro-2,1,3-benzoxadiazol-4-yl)thio]ethyl-4-methoxybenzoate;
5-[4-(tert-butyl)-1,3-thiazol-2-yl]-2,1,3-benzoxadiazole;
N-benzyl-7-chloro-4-nitro-2,1,3-benzoxadiazol-5-amine;
4-nitro-7-(phenylmethylsulfanyl)-2,1,3-benzoxadiazole;
4-nitro-7-(phenylmethylsulfanyl)-2,1,3-benzoxadiazole;
2-(hydroxymethyl)-5-[6-[(4-nitro-2,1,3-benzoxadiazol-7-yl)sulfanyl]purin-9-yl]oxolane-3,4-diole;
2-[2-amino-6-[(4-nitro-2,1,3-benzoxadiazol-7-yl)sulfanyl]purin-9-yl]-5-(hydroxymethyl) oxolane-3,4-diol; and
physiologically tolerable salts, solvates, and physiologically functional derivatives thereof.

10. The method of claim 8, wherein the subject is selected from the group consisting of: humans; mammals; and birds.

11. A method for inhibiting the growth of a virus selected from the group consisting of: influenza A virus, influenza B virus and respiratory syncytial virus, comprising contacting the virus with a 2,1,3-benzoxadiazole compound.

12. The method of claim 11, wherein the 2,1,3-benzoxadiazole compound is selected from the group consisting of:
4-[(4-methoxybenzyl)thio]-7-nitro-2,1,3-benzoxadiazole;
2-[(7-nitro-2,1,3-benzoxadiazol-4-yl)thio]ethyl 4-methoxybenzene-1-sulfonate;
4-[(4-methylphenyl)thio]-7-nitro-2,1,3-benzoxadiazole;
4-[(2,4-dichlorophenyl)thio]-7-nitro-2,1,3-benzoxadiazole;
2-[(7-nitro-2,1,3-benzoxadiazol-4-yl)thio]ethan-1-ol;
4-[(4-methylbenzyl)thio]-7-nitro-2,1,3-benzoxadiazole;
4-[(4-fluorophenyl)thio]-7-nitro-2,1,3-benzoxadiazole;
4-[(3-chlorophenyl)thio]-7-nitro-2,1,3-benzoxadiazole;
2-[(7-nitro-2,1,3-benzoxadiazol-4-yl)thio]ethyl-4-methoxybenzoate;
5-[4-(tert-butyl)-1,3-thiazol-2-yl]-2,1,3-benzoxadiazole;
N-benzyl-7-chloro-4-nitro-2,1,3-benzoxadiazol-5-amine;
4-nitro-7-(phenylmethylsulfanyl)-2,1,3-benzoxadiazole;
4-nitro-7-(phenylmethyl sulfonyl)-2,1,3-benzoxadiazole;
2-(hydroxymethyl)-5-[6-[(4-nitro-2,1,3-benzoxadiazol-7-yl)sulfanyl]purin-9-yl]oxolane-3,4-diole;
2-[2-amino-6-[(4-nitro-2,1,3-benzoxadiazol-7-yl)sulfanyl]purin-9-yl]-5-(hydroxymethyl) oxolane-3,4-diol; and
physiologically tolerable salts, solvates, and physiologically functional derivatives thereof.

13. A pharmaceutical composition comprising a compound selected from the group consisting of:
4-[(4-methoxybenzyl)thio]-7-nitro-2,1,3-benzoxadiazole;
2-[(7-nitro-2,1,3-benzoxadiazol-4-yl)thio]ethyl 4-methoxybenzene-1-sulfonate;
4-[(4-methylphenyl)thio]-7-nitro-2,1,3-benzoxadiazole;
4-[(2,4-dichlorophenyl)thio]-7-nitro-2,1,3-benzoxadiazole;
2-[(7-nitro-2,1,3-benzoxadiazol-4-yl)thio]ethan-1-ol;
4-[(4-methylbenzyl)thio]-7-nitro-2,1,3-benzoxadiazole;
4-[(4-fluorophenyl)thio]-7-nitro-2,1,3-benzoxadiazole;
4-[(3-chlorophenyl)thio]-7-nitro-2,1,3-benzoxadiazole;
2-[(7-nitro-2,1,3-benzoxadiazol-4-yl)thio]ethyl-4-methoxybenzoate;
5-[4-(tert-butyl)-1,3-thiazol-2-yl]-2,1,3-benzoxadiazole;
N-benzyl-7-chloro-4-nitro-2,1,3-benzoxadiazol-5-amine;
4-nitro-7-(phenylmethylsulfanyl)-2,1,3-benzoxadiazole;
4-nitro-7-(phenylmethylsulfonyl)-2,1,3-benzoxadiazole;
2-(hydroxymethyl)-5-[6-[(4-nitro-2,1,3-benzoxadiazol-7-yl)sulfanyl]purin-9-yl]oxolane-3,4-diole;
2-[2-amino-6-[(4-nitro-2,1,3-benzoxadiazol-7-yl)sulfanyl]purin-9-yl]-5-(hydroxymethyl) oxolane-3,4-diol; and
physiologically tolerable salts, solvates, and physiologically functional derivatives thereof.

14. The pharmaceutical composition of claim 13, wherein the composition further comprises at least one excipient.
